(19) United States
(12) Patent Application Publication
(10) Pub. No.: US 2023/0321436 A1
(43) Pub. Date: Oct. 12, 2023

(54) MEDICAL DEVICE FOR STIMULATING NEURONS OF A PATIENT TO SUPPRESS A PATHOLOGICALLY SYNCHRONOUS ACTIVITY THEREOF

(71) Applicant: GRETAP AG, Zug (CH)

(72) Inventor: Peter Alexander Tass, Tegernsee (DE)

(73) Assignee: GRETAP AG, Zug (CH)

(21) Appl. No.: 18/131,659

(22) Filed: Apr. 6, 2023

Related U.S. Application Data
(63) Continuation of application No. PCT/EP2021/077619, filed on Oct. 6, 2021.
(60) Provisional application No. 63/087,906, filed on Oct. 6, 2020.

(51) Int. Cl. A61N 1/36

(52) U.S. Cl. CPC A61N 1/36025; A61N 1/36031; A61N 1/36034

(57) ABSTRACT

The present invention refers to a medical device (10) for stimulating neurons of a patient to suppress a pathologically synchronous activity of the neurons. The medical device (10) comprises a non-invasive therapeutic stimulation unit (12) configured to administer a plurality of stimuli to a patient's body which is configured to suppress the pathologically synchronous activity; a clock unit (24) configured to generate at least one periodic or cyclical clock signal (S(t); S′(t); S(t), S′(t)) related to a physiological activity or physiological cycle of the patient; and a control unit (18) configured to control operation of the medical device (10) in dependence on the at least one clock signal (S(t); S′(t); S(t), S′(t)).

### CROSS-REFERENCE TO RELATED APPLICATIONS

This application is continuation of PCT International Patent Application No. PCT/EP2021/077619, filed Oct. 6, 2021, which claims benefit of priority to U.S. Patent Application No. 63/087,906, filed Oct. 6, 2020. The contents of each of the aforementioned applications are incorporated herein by reference in their entirety.

### TECHNICAL FIELD

The invention relates to a medical device for stimulating neurons of a patient to suppress a pathologically synchronous activity of the neurons.

### TECHNOLOGICAL BACKGROUND

Several brain disorders, such as Parkinson's disease, are characterized by an abnormally strong synchronous activity of a neuronal population, i.e. strongly synchronized neuronal firing or bursting. Besides Parkinson's disease, this may also apply, for example, to essential tremor, dystonia, dysfunction after stroke, epilepsy, depression, migraine, tension headache, obsessive-compulsive disorder, irritable bowel syndrome, chronic pain syndromes, pelvic pain, tinnitus, dissociation in borderline personality disorder and post-traumatic stress disorder. As such, abnormally synchronized neuronal activity is of great relevance for several neurological and psychiatric disorders.

The pharmacological treatment for Parkinson's disease with, for example, L-DOPA may have limited therapeutic effects and may cause significant long-term side effects. High-frequency Deep Brain Stimulation (DBS) for Parkinson's disease is a standard for medically refractory patients in advanced stages of the disease. However, DBS requires surgical procedures associated with a significant risk. For instance, depth electrode implantation in dedicated target areas in the brain may cause bleedings. Furthermore, standard continuous high- frequency DBS may cause side effects.

Further, non-invasive treatment approaches are known which apply vibrotactile multichannel stimulation to counteract Parkinsonian signs or other neurological and psychiatric disorders caused by an abnormally synchronized neuronal activity. Specifically, according to one approach, periodic stimulations are administered to a patient which are intended to selectively activate at least a part of the patient's neurons affected by the abnormally synchronized activity. This stimulation may be performed according to a relatively simple and repeating actuation pattern which specifies what kind of stimuli are administered to the patient and at which time. According to a further known approach, the non-invasive stimulation may be carried out according to a more complex actuation pattern which may continuingly vary during and among treatments as described, for example, in WO 2016/207247 A1 and WO 2019/243634 A1. Further, a stimulation technique referred to as “Coordinated reset” (CR) is known which applies characteristic sequences of brief stimuli administered to different subpopulations within an abnormally synchronized neural network. By applying these stimulation techniques, a desynchronization of the targeted neural network may be restored and maintained.

Typically, for treating such abnormally synchronized neuronal activities, the known non-invasive stimulation treatments are applied frequently, e.g. in sessions for several hours every day during weeks or months. However, it has been found that the quality of the therapeutic effect may vary among the different treatment sessions, which thereby may affect the overall efficiency of the treatment.

### SUMMARY OF THE INVENTION

In view of the technical background, it is an objective of the present invention to provide an is improved non-invasive medical device for stimulating neurons of the patient which enables to provide effective therapeutic treatments to suppress a pathologically synchronous activity of the patient's neurons.

This objective is solved by means of a medical device with the features of independent claim 1.

Accordingly, a medical device for stimulating neurons of a patient to suppress a pathologically synchronous activity of the neurons is provided. The medical device comprises a non-invasive therapeutic stimulation unit configured to administer a plurality of stimuli to a patient's body. The plurality of stimuli is configured to suppress the pathologically synchronous activity when being administered to the patient's body. The medical device further comprises a clock unit configured to generate at least one clock signal, in particular a periodic or cyclical clock signal, related to a physiological activity or physiological cycle of the patient, and a control unit configured to control operation of the medical device in dependence on the at least one clock signal.

The proposed medical device may be used for therapeutic and diagnostic purposes. Specifically, the proposed medical device may be intended to be used for the treatment of neurological or psychiatric diseases, in particular, Parkinson's disease, essential tremors, dystonia, etc. The medical device may also be used for the treatment of other neurological or psychiatric diseases, such as epilepsy, tremors as a result of Multiple Sclerosis as well as other pathological tremors, depression, movement disorders, diseases of the cerebellum, obsessive compulsive disorders, Tourette syndrome, functional disorders following a stroke, spastics, tinnitus, sleep disorders, schizophrenia, irritable bowel syndrome, addictive disorders, personality disorders, attention deficit disorder, attention deficit hyperactivity syndrome, gaming addiction, neuroses, eating disorders, burnout syndrome, fibromyalgea, migraine, cluster headache, general headaches, neuronalgia, ataxy, tic disorder or hypertension, and also for the treatment of other diseases.

The aforementioned diseases can be caused by a disorder of the bioelectric communication of groups of neuronal cells which are connected to one another in specific circuits. Hereby, a 10 neuron population generates a continuous pathological neuronal activity and a pathological connectivity (network structure) possibly associated therewith. In this respect, a large number of neurons form synchronous action potentials, this means that the concerned neurons fire or burst excessively synchronously. In addition, the pathological neuron population may have an oscillating or intermittent neuronal activity, this means that the neurons fire or burst rhythmically or intermittently. In the case of neurological or psychiatric diseases, the mean frequency of the pathological rhythmic activity of the concerned groups of neurons approximately may be in the range of 1 Hz to 60 Hz, particularly in the range of 1 Hz to 30 Hz, but may also be outside of this range. By contrast, the neurons of healthy people fire or burst qualitatively differently, for example, in an uncorrelated manner.

In other words, each of the aforementioned diseases may be characterized by at least one neuronal population in the brain or spinal cord of the patient which has a pathological synchronous neuronal activity. For suppressing such a pathologically synchronous activity, the proposed medical device may be configured to stimulate the affected neuronal population so as to cause the affected neuronal population to fire or burst in an uncorrelated manner, i.e. non-synchronously.

Specifically, the medical device is a non-invasive therapeutic treatment device. This means that the medical device deploys a non-invasive procedure to achieve the intended therapeutic effect. In other words, in an operational state, the medical device is not implanted into the patient's body and, hence, does not require a skin incision.

For acting upon the patient and thus for achieving the intended therapeutic effect, the medical device comprises the non-invasive therapeutic stimulation unit, also referred to as the “stimulation unit” in the following. The stimulation unit is configured to administer or induce the plurality of stimuli, i.e. different stimuli, to the patient's body.

In the context of the present disclosure, the terms “stimulus” and “stimuli” refer to any information or event generated by the stimulation unit which can be sensed by the patient, i.e. registered by the patient's senses. Such stimuli may have the modality of, for example, acoustic stimuli, tactile stimuli, vibratory stimuli, vibrotactile stimuli, visual stimuli, electrical stimuli, in particular electrical tongue stimuli, and/or thermal stimuli. In other words, the plurality of stimuli generated by the stimulation unit may comprise at least one of an acoustic stimulus, a tactile stimulus, a vibratory stimulus, a vibrotactile stimulus, a visual stimulus, an electrical stimulus, in particular an electrical tongue stimulus, and/or a thermal stimulus. These stimuli, when being administered to the patient's body, may be sensed by receptors, for example, in the eyes, the ears and/or the skin of the patient depending on the stimuli's modality and are guided from there to a patient's nerve system causing an activation or stimulation of neurons in the patient's brain or spinal cord.

Thus, for stimulating the neuronal population affected by the pathologically synchronous activity, the stimulation unit is configured to administer the plurality of stimuli to the patient's is body which, upon being sensed by respective receptors and guided to the patient's nerve system, may cause activation of at least a part of the affected neuronal population. For doing so, i.e. for causing stimulation of the affected neuronal population, the characteristics of the different stimuli generated by the stimulation unit may be selectively set, particularly in view of their modality, intensity, amplitude, frequency, etc.

The stimulation unit may comprise at least one stimulation element which is configured to, upon actuation, generate at least one stimulus. For example, the stimulation element may be configured to generate vibratory and/or tactile and/or vibrotactile stimuli. For doing so, the stimulation element may comprise an electro-mechanical actuator for converting electrical energy into a movement of a component, such as a rod, which may be configured to mechanically act upon the patient's skin. In such a configuration, the electro-mechanical actuator may be provided in the form of an equal current motor, a voice coil, a piezo-electric transducer or a transformer built up of electro-active polymers which change their shape on the application of an electric current.

Alternatively or additionally, the stimulation unit may comprise at least one stimulation element which is configured to generate acoustic stimuli. For example, such a stimulation element may be provided in the form of a loudspeaker configured to selectively and/or variedly generate tones at a desired frequency and at a desired volume level. According to one configuration, a stimulation unit equipped with such an acoustic stimulation element may comprise or be provided in the form of headphones for the patient.

Alternatively or additionally, the stimulation unit may comprise at least one stimulation element which is configured to generate visual or optical stimuli. For example, such a stimulation element may be provided in the form of or may comprise at least one light source, e.g. in the form of a light-emitting diode, configured to selectively emit light of a desired frequency and at a desired intensity level or brightness. According to one configuration, the stimulation unit may be integrated into glasses configured to be put on by the patient. In this respect, the stimulation unit may comprise stimulation elements which are configured to affect the light guided into the patient's eyes to generate the different stimuli. The stimulation elements may be provided in the form of light sources for emitting light and/or shutter, filter and/or deflecting elements for affecting the light to be guided into the patient's eyes.

For enabling effective treatment of the pathologically affected neuronal population, the stimulation unit may be configured to selectively and intermittently administer the plurality of stimuli. Accordingly, the different stimuli may be generated at defined intervals and/or at defined locations.

Alternatively or additionally, the stimulation unit may be configured to administer a periodic stimulation constituted by the plurality of stimuli. That is, the stimulation unit may be configured to generate the plurality of stimuli according to a regular and repeating actuation and timing pattern. In a further development, the different stimuli may be administered according to an actuation and timing pattern which aims at mutually time-shifted actuations of neuronal sub-populations of varying composition, i.e. in terms of location and quantity. For doing so, an actuation sequence defined by the actuation and timing pattern may be variedly adjusted during operation, for example, in view of an order and characteristic of stimuli to be administered and/or a number and combination of stimuli to be simultaneously administered.

The general functional and structural configuration of such a stimulation unit for suppressing a pathological synchronous activity of the patient's neurons as well as its actuation are well known to a person skilled in the art and are therefore not further specified in the present disclosure. In this context, explicit reference is made to, for example, WO 2019/243634 A1 and WO 2020/049004 A1 in which the structural configuration and actuation of such a stimulation unit is specified. Rather, technical features interlinked with the present invention are specified in the following, in particular in view of the clock-signal-dependent control of the medical device.

As set forth above, the medical device comprises the clock unit which is configured to generate the at least one clock signal, in particular the at least one periodic or cyclical clock signal. In the context of the present disclosure, the term “periodic signal” refers to a signal which course or values repeat/s, in particular substantially repeat/s, at regular, in particular substantially regular, time intervals. Further, the term “cyclical signal” refers to a signal which comprises a repeating order of events, i.e. events that repeat themselves regularly in the same order and optionally in a regularly repeated period of time. Accordingly, the clock signal may comprise or be constituted by a repeating sequence of at least two subsequent phases.

According to the proposed medical device, the clock signal, which is used to control operation of the medical device, is related to a physiological activity or a physiological cycle of the patient. In the context of the present disclosure, the term “physiological activity” refers to an activity of the patient which induces a measurable change of a patient's physiological condition. Further, the term “physiological cycle” refers to a sequence of regularly repeating modifications in physiological conditions or activities of the patient.

The clock signal is related to a physiological activity or physiological cycle. That is, the clock signal may correlate or be indicative of a physiological activity and/or physiological cycle. In other words, based on the clock signal, characteristics of a physiological activity or a physiological cycle may be derived. For example, the clock signal may be indicative of characteristics and/or the course over time of a reference physiological activity or reference physiological cycle. The terms “reference physiological activity” and “reference physiological cycle” refer to events which serve as or relate to a reference state of the patient, in particular a target or desired reference state, i.e. a reference state to be attained. Alternatively or additionally, the clock signal may be indicative of characteristics and/or the course over time of an actual physiological activity or actual physiological cycle. Accordingly, the terms “actual physiological activity” and “actual physiological cycle” refer to a present or prevailing state of the patient.

The control unit is configured to control operation of the medical device in dependence on the at least one clock signal. By doing so, a medical device is proposed, the operation and actuation of which is locked to a physiological activity or physiological rhythm of the patient.

Thus, a medical device is provided which is controlled in a physiological-activity-dependent or physiological-rhythm-dependent manner.

In various aspects regarding, for example, brain activity, metabolic activity and perfusion, the state and condition of a patient or human subject varies continuously. For instance, plasticity mechanisms and parameters may vary in time. In the context of the present invention, it has been found that the effect of therapeutic stimulation may depend on a patient's actual state and condition. Hence, to improve efficacy of therapeutic stimulation, the medical device is proposed which is actuated in dependence on and thus locked to a physiological activity or physiological rhythm of the patient. For doing so, the proposed medical device is equipped with the control unit which is configured to control operation of the medical device in dependence on the at least one clock signal. In this way, the proposed solution enables to actuate the medical device in a patient's-state-dependent manner, thereby allowing that a stimulation of an affected neuronal population is carried out when a condition or state of the patient is prevailing which is favorable for the therapeutic treatment and/or during which a significant success of the therapeutic treatment can be expected.

According to one embodiment, the medical device may be provided so as to be actuated in dependence of a physiological activity, in particular in dependence of movements of bodily parts. Accordingly, the clock signal generated by the clock unit may relate to or be indicative of movements of bodily parts. Such movements may be rhythmic and/or oscillatory and/or intermittent and/or occasional movements. Thus, for generating the clock signal, the clock unit may comprise or be communicatively connected to a sensor unit for measuring or determining movements of bodily parts as described further below.

The proposed medical device may be provided so as to be locked to or actuated in dependence on a physiological cycle corresponding to a relatively slow physiological rhythm, e.g. is having a frequency in the range of around 0.03 Hz to 0.5 Hz, in particular of 0.1 Hz to 0.35 Hz. Physiological cycles with a slow physiological rhythm have been identified as favorable for the therapeutic treatment effect. However, the present invention and the effects interlinked therewith are not limited to such slow physiological cycles.

Specifically, such a slow physiological cycle may be a breathing cycle. Thus, according to one embodiment, the physiological cycle underlying the clock signal, i.e. to which the clock signal relates, may be a breathing cycle of or for the patient. Specifically, the breathing cycle may be divided into different subsequent phases, preferably in at least one of an inhalation phase, a breath holding phase and an exhalation phase. In a further development, the breathing cycle may be constituted by and divided into the subsequent phases of inhalation phase, breath holding phase, exhalation phase and pause phase, particularly in that order. Alternatively, the breathing cycle may be constituted by and divided into the subsequent phases of inhalation phase and exhalation phase. Generally, the physiologic activity, to which the clock signal may relate, may refer to at least one of the different phases of the physiological cycle.

Alternatively or additionally, the physiological cycle, i.e. to which the clock signal relates, may be a blood pressure and/or blood circulation and/or skin microcirculation cycle. Specifically, the physiological cycle may relate to a blood volume change cycle in a tissue bed, in particular a microvascular tissue bed.

Alternatively or additionally, the physiological cycle, i.e. to which the clock signal relates, may relate to oscillations of skin microcirculation. Such physiological cycle may have a cycle frequency of, for example, around 0.1 Hz and may be measured or recorded by means of a photoplethysmography, in particular infra-red photoplethysmography.

The clock signal may define a relation between two sets of values, particularly a first set of values quantifying a characteristic of the physiological activity or the physiological cycle and a second set of values relating to time. For example, the first set of values may be defined such that each value may refer to one phase of the physiological cycle, respectively. In this way, the clock signal may define corresponding data sets associating a phase of the physiological cycle to a point in time. In this way, the clock signal may indicate which phase of the physiological cycle is associated to which period of time.

Alternatively, the first set of values may quantify an amplitude of a parameter or characteristic being indicative of the physiological activity of physiological cycle, such as a flow rate of gases being supplied to or discharged from the patient's lungs or a change of the patient's lung volume. As such, the clock signal may indicate a course of the amplitude, i.e. the considered physiological characteristic, over time. For example, the amplitude may be indicative of a breathing flow rate, a breathing-related chest wall motion, and/or breathing-related changes in light absorption at a region of the patient's skin illuminated and measured by a pulse oximeter. Further, when the physiological cycle refers to a blood pressure cycle in a tissue bed, the amplitude may be indicative of a blood-pressure-cycle- and/or blood-circulation-cycle-induced change in light absorption at a region of the patient's skin which may be illuminated and measured by a pulse oximeter. Sections of the clock signal, i.e. the course of the amplitude over time, may be associated to individual physiological activities or to individual phases of the physiological cycle. In other words, sections or parts of the clock signal, in particular in the course of the amplitude over time, which follow or coincide or are similar to a predetermined pattern may be associated to individual physiological activities and/or to individual phases of the physiological cycle.

For example, sections of the clock signal during which the amplitude has a negative value or during which the amplitude is maintained below a negative threshold value for a predetermined period of time may be associated to a first phase, in particular the exhalation phase, for example when the clock signal may be indicative of a change of the patient's lung volume. Alternatively or additionally, sections of the clock signal during which the amplitude has a positive value or during which the amplitude is maintained above a positive threshold value for a predetermined period of time may be associated to a second phase, in particular the inhalation phase. Alternatively or additionally, sections of the clock signal during which the amplitude is maintained in a range around zero or in a range between the negative and the positive threshold values for a predetermined period of time may be associated to a further phase, in particular the breath holding phase and/or the pause phase. Specifically, sections of the clock signal during which the amplitude changes from a value equal to the positive threshold value to a value equal to the negative threshold value and during which the amplitude is maintained in a range around zero or in a range between the negative and the positive threshold values for a predetermined period of time may be associated to a third phase, in particular the breath holding phase, and sections of the clock signal during which the amplitude changes from a value equal to the negative threshold value to a value equal to the positive threshold value and during which the amplitude is maintained in a range around zero or in a range between the negative and the positive threshold values for a predetermined period of time may be associated to a fourth phase, in particular the pause phase.

According to a further development, it has been found that it may be favorable to put the patient into a reference condition when he/she is subjected to a treatment with the medical device. As to substance, by putting the patient to be treated into the reference state, the medical device may be more precisely adapted to the physiological characteristics of the patient without requiring to monitor or measure prevailing physiological characteristics since the device is may be pre-adjusted to the reference condition. In this way, reproducibility of the intended therapeutic effect may be ensured. Furthermore, the reference condition may refer to a condition of the patient in which an increased or improved therapeutic effect is to be expected. For example, such a reference condition may refer to a condition in which the patient applies slow deep breathing which may induce favorable states in a reliable manner. Also, the reference condition as such may have a therapeutic effect on the pathologically synchronous activity of the patient's neurons. This may particularly be the case when the patient applies slow deep breathing which, e.g., may have several beneficial effects on Parkinson's disease or other neurological and psychiatric disorders.

For putting the patient into the reference condition, the medical device may comprise a pacing unit configured to provide guidance for the patient to be treated. Specifically, the pacing unit may be configured to provide guidance to the patient by instructing the patient on how to perform a physiological activity in order to reach the intended reference state. In other words, the pacing unit may be configured to provide guidance for a physiological activity or physiological cycle of the patient to put the patient into the reference state. For doing so, the control unit of the medical device may be configured to control operation of the pacing unit, i.e. to actuate the pacing unit, in dependence on a reference clock signal, which may also be referred to as an instructed clock signal or a target clock signal. The reference clock signal preferably is related to the reference physiological activity and/or the reference physiological cycle.

According to one embodiment, the pacing unit may be configured to instruct the patient to perform slow deep breathing. Accordingly, the control unit may be configured to control the pacing unit in dependence on a reference clock signal which may relate to a slow breathing cycle. For doing so, the cycle length and cycle frequency may lie within a slow breathing range, for example, in a range of about 0.07 Hz to 0.16 Hz. Further, the length of the different phases of the physiological cycle may be in an associated range relating to slow deep breathing, respectively. For example, the phase length of at least one of the inhalation phase, the breath holding phase, the exhalation phase and the pause phase may lie within the range of 2.5 s to 5 s or in the range of 1.8 s to 3.6 s, respectively. The individual phases of the cycle may differ in view of their duration relative to one another or may have the same duration or length. For example, according to one configuration, the inhalation phase and breath holding phase may have a corresponding or identical duration, whereas the exhalation phase may be twice as long as the inhalation phase.

For providing guidance to and thus for instructing the patient to be treated, the pacing unit is configured to provide information to the patient. For doing so, the pacing unit may be configured to convert information provided by the clock unit and/or the control unit of the medical device into a form more accessible by the patient. For doing so, the pacing unit may be configured to convert or extract information from the clock signal which are then presented or provided to the patient by an output device, such as a display or loudspeaker.

Specifically, for providing guidance to the patient, the pacing unit may be configured to provide non-invasive guidance stimuli to the patient. These guidance stimuli may be configured to provide information to the patient, rather than suppressing the pathologically synchronous neuronal activity. However, in some configurations, the guidance stimuli may also contribute to the suppression of the pathologically synchronous activity of the neurons. In particular, as the guidance stimuli, the pacing unit may be configured to provide at least one of visual stimuli, acoustic stimuli, tactile stimuli, vibratory stimuli, vibrotactile stimuli, electrical stimuli, transdermal electrical stimuli transdermal magnetic stimuli, transcranial electrical stimuli, transcranial magnetic stimuli and electrical tongue stimuli. For doing so, the pacing unit, in particular its output device, may comprise at least one further stimulation unit which, for example, may be provided in the form of a display unit for visualizing the reference clock signal, i.e. by providing visual stimuli. The display unit may be part of a mobile device, such as a smart phone.

Alternatively or additionally, besides or instead of the pacing unit, also the non-invasive therapeutic stimulation unit may be configured to provide guidance to the patient. For doing so, the stimulation unit may be configured to further generate the above described guidance stimuli for providing guidance for a physiological activity of the patient and thus to instruct the patient during operation. For example, the medical device may be configured such that, each time the stimulation unit is actuated, i.e. administers stimuli to the patient's body, the patient is instructed to perform a physiological activity related thereto, e.g. is instructed to inhale. By such a configuration, the stimuli generated by the stimulation unit may constitute both therapeutic stimuli, i.e. which contribute to the suppression of the pathologically synchronous activity of the neurons, and guiding stimuli for instructing the patient. In this way, guidance may be provided to the patient without requiring to equip the medical device with an additional stimulation unit or pacing unit. In other words, in such a configuration, the non-invasive therapeutic stimulation unit may also constitute a pacing unit. Accordingly, the stimulation unit may be actuated by the control unit in dependence on the reference clock signal.

The guidance stimuli may be associated to at least one phase of the physiological cycle of the patient. Further, the guidance stimuli may be configured to guide or instruct the patient to perform a physiological activity, in particular a physiological activity which is related to the associated phase. For example, the guidance stimuli may be associated to at least one phase of the physiological cycle and may be configured to instruct a patient to perform inhalation during the associated phase or phases.

According to one configuration, the control unit may be configured to actuate the stimulation unit in dependence on a predetermined actuation pattern, which in particular may not be dependent on the clock signal generated by the clock unit. In this configuration, only the pacing unit may be controlled in dependence on the clock signal, i.e. the reference clock signal. In other words, the stimulation unit is controlled in a physiological cycle non-dependent manner. Since the pacing unit provides guidance to the patient to be treated based on the clock signal, the medical device may ensure that the patient is put into a favorable reference state, thereby enabling to effectively perform the therapeutic treatment.

Alternatively, the control unit may be configured to actuate the stimulation unit in dependence on the clock signal. According to one configuration, the control unit may be configured to actuate the stimulation unit in dependence on the reference clock signal. In this way, the stimulation unit may be actuated in a manner dependent on the reference physiological activity or the reference physiological cycle.

In a further development, the medical device may comprise a sensor unit configured to monitor the actual physiological activity or the actual physiological cycle of the patient. Further, the clock unit may be configured to generate the clock signal in dependence on monitoring data obtained by the sensor unit. The thus generated clock signal may also be referred to as “recorded clock signal”. The recorded clock signal thus may be indicative of the actual physiological activity or the actual physiological cycle. In other words, the clock unit is configured to use the monitoring data obtained by the sensor unit as an input for generating the recorded clock signal. The monitoring data obtained by the sensor unit may be transmitted wirelessly or via a cable connection to the clock unit.

As regards the sensor unit, any sensor unit may be used which is capable of measuring and monitoring changes of the patient's physiological condition and/or physiological activity and/or physiological cycle. For example, in case the physiological activity and/or physiological cycle refers to a breathing cycle, the sensor unit may be or comprise a flowmeter configured to measure and monitor a flow rate of gasses which are directed into and discharged from a patient's lungs. Alternatively or additionally, the flowmeter may be configured to measure and monitor concentration or partial pressure of carbon dioxide in the respiratory gases of the patient, e.g. by means of capnography techniques. Specifically, the flowmeter may be attached to a mask of the sensor unit which is configured to be releasably attached to a patient's head. Alternatively or additionally, the sensor unit may be or comprise at least one body surface sensor which may be configured to monitor breathing-related chest wall motion. For doing so, the at least one body surface sensor may be equipped with at least one of an accelerometer, a gyroscope, a magnetometer, a strain gauge, an elastic band with electrical stretching-dependent resistance and an optical fiber with curvature-depending light transmission. Alternatively or additionally, the sensor unit may be or comprise a pneumograph which is configured to measure and monitor velocity and force of chest movements, for example, by measuring changes in an electrical impedance between two or more electrocardiography electrodes. Alternatively or additionally, the sensor unit may comprise at least one piezoelectric sensor which may be configured to be attached to the patient's chest for measuring and monitoring chest movements of the patient.

Alternatively or additionally, the sensor unit may comprise at least one sensor configured to monitor changes in light absorption at a region of the patient's skin which may be illuminated and measured by a pulse oximeter. Such a sensor unit may be a photoplethysmogram (PPG) sensor which enables to detect blood volume changes in a microvascular tissue bed. It has been found that skin blood flow is indicative of breathing activities. Accordingly, the PPG sensor can be used to monitor breathing. Hence, a PPG sensor conveniently placed at the ear, fingertip, nasal septum or forehead of the patient may be used to measure breathing events and a breathing rhythm of the patient. Alternatively or additionally, the sensor unit may comprise a laser Doppler flowmetry sensor configured to measure cardiovascular slow oscillations. In this regard, it has been found that cardiovascular slow oscillations are related to breathing-modulated skin blood perfusion and therefore are indicative of the breathing activity and rhythm.

Alternatively or additionally, the sensor unit may be configured to determine and/or quantify movements of bodily parts based on which the clock unit generates the clock signal. Specifically, the sensor unit may be configured to measure quantities related to movements of bodily parts. For doing so, the sensor unit may comprise, for example, at least one goniometer which in particular may be configured to measure angles between two sites of the patient's body. In one configuration, the sensor unit may comprise at least one goniometer, in particular an electro-goniometer, which is wearable, for example, in the region of bodily joints, such as elbows, knees, etc. Alternatively or additionally, the sensor unit may comprise at least one accelerometer attached to the patient's body, in particular to bodily part's, the movement of which is to be measured. The accelerometer may be configured to measure quantities related to an acceleration of the bodily part, the movement of which is to be monitored. Alternatively or additionally, the sensor unit may comprise at least one gyroscope attached to the patient's body, in particular to bodily part's, the movement of which is to be measured. The gyroscope may be configured to measure quantities related to an angular velocity of the bodily part.

In the context of a further development of the present invention, it has been found that adapting the delivery of therapeutic stimulation to a physiological activity and/or physiological cycle may have favorable effects on the therapeutic treatment provided by the medical device. This applies to both a reference physiological activity and/or cycle as well as an actual physiological activity and/or cycle.

Thus, for adapting the delivery of therapeutic stimulation to a reference condition or an actual condition of the patient, the control unit may be configured to actuate the stimulation unit in dependence on the at least one clock signal generated by the clock unit. More specifically, the control unit may be configured to actuate the stimulation unit in dependence on the reference clock signal to adapt the delivery of stimulation to the reference condition and/or in dependence on the recorded clock signal to adapt the delivery of stimulation to the recorded or actual condition of the patient. In this way, a physiological-condition-dependent stimulation may be effected. In a further development, the control unit may be configured to control operation of the stimulation unit such that stimulation parameters, e.g. characteristics of the plurality of stimuli, such as intensity, vibration frequency, amplitude, etc., are adjusted and controlled in dependence on the clock signal.

Further, the control unit may be configured to set different operating modes of the medical device in dependence on the clock signal and thus in dependence on the physiological activities or the physiological cycle, in particular in dependence on at least one phase of the physiological cycle. That is, individual operating modes may be scheduled or activated in dependence on physiological activities or at least one phase of the physiological cycle. Specifically, the beginning and/or the end of an operating mode may coincide with the beginning and/or the end of a physiological activity or a phase of a physiological cycle. Alternatively or additionally, the beginning and/or the end of an operating mode may be time-shifted relative to the beginning and/or the end of a physiological activity or a phase of a physiological cycle. Alternatively or additionally, a duration of an operating mode may be equal to a duration of a physiological activity or a phase of a physiological cycle associated thereto. Alternatively or additionally, a duration of an operating mode may be different to a duration of a physiological activity or a phase of a physiological cycle associated thereto.

The control unit may be configured to detect the different phases in the cyclical clock signal. For doing so, the control unit may apply means of data analysis, such as filtering, in particular bandpass filtering, Hilbert transformation, transformation of the clock signal in a breathing pattern circle, statistical evaluation and/or linear or non-linear approximation, etc. Further, the control unit may be configured to compare the clock signal, i.e. the course of its amplitude over time, with an upper and lower threshold as described above to identify different phases of the physiological cycle and the clock signal. In this regard, the control unit may associate sections of the clock signal during which the amplitude is above the upper threshold to a first phase, such as the inhalation or exhalation phase, and/or sections of the clock signal during which the amplitude is below the lower threshold to a second phase, in particular the exhalation or inhalation phase, and/or sections of the clock signal during which the amplitude is around zero or between the upper and lower threshold to a further phase, in particular the is breath holding phase or pause phase.

Further, the control unit may be configured to operate the stimulation unit in a first operating mode during a first phase of the clock signal and in a second mode during a second phase of the clock signal, in particular the reference clock signal or recorded clock signal. In this way, a cycle-dependent stimulation may be provided. The first and the second operating mode of the stimulation unit may differ from one another.

Specifically, the first and the second operating mode may differ in view of stimuli which are to be administered during the respective operating mode. For example, the first and the second operating mode may differ from one another in at least one of a stimulation modality, a frequency of the stimuli to be administered, type and number of stimulation elements to be actuated, a stimulation pattern according to which stimulation elements are to be actuated, a stimulation region at the patient's body to which the stimuli are to be administered, stimuli amplitude, stimuli intensity, etc. According to one configuration, the stimulation modality of the stimuli generated during the first and the second operating mode may differ such that, for example, vibratory or tactile or vibrotactile stimuli are generated during the first operating mode and acoustic stimuli are generated during the second operating mode. Further, the type and number of stimulation elements may vary among the first and the second operating mode such that different stimulation elements are to be actuated in the first operating mode compared to the second operating mode. Alternatively or additionally, different stimulating elements may be actuated or different regions on the patient's body may be stimulated among the different operating modes. For example, fingertips of the patient's left hand may be stimulated in the first operating mode and fingertips of the patient's right hand may be stimulated in the second operating mode. Alternatively or additionally, stimulation parameters or characteristics may vary among the different operating modes of the stimulation unit. For example, the stimuli provided by the stimulation unit may be provided at a higher intensity or amplitude level during the first operating mode compared to the second operating mode, and vice versa.

According to one configuration, the control unit may be configured to control the stimulation unit such that, in the first operating mode, it generates the plurality of stimuli to be administered the patient's body and, in the second operating mode, it stops generation of the plurality of stimuli. In other words, in the second operating mode, the patient may be prevented from being subjected to stimuli generated by the stimulation unit. Accordingly, therapeutic stimulation provided by the stimulation unit may only be delivered during a part of two or more different operating modes.

According to one configuration, the first phase and the second phase of the clock signal may relate or be associated to different phases of a breathing cycle. Each one of the first and the is second phase may relate to one or more breathing cycle phases, wherein the one or more breathing cycle phase associated to the first phase may be different compared to those associated to the second phase. For example, the first phase may comprise or relate to the inhalation phase or the exhalation phase, while the second phase may comprise or relate to the exhalation phase or the inhalation phase.

In this way, an inhalation and exhalation dependent delivery of therapeutic stimulation may be provided which may particularly contribute to an effective therapeutic treatment. The reason for this is that the human respiratory center forms wide-spread afferent projections (synaptic connections) with numerous brain regions. As such, the respiratory center modulates the activity of these brain regions depending on the phase of the breathing cycle. For instance, breathing-induced modulatory effects may affect various distinct brain areas throughout the entire brain, related to various behavioral, cognitive and emotional states, and may specifically differ between inhalation and non-inhalation phases.

Further, the control unit may be configured to assess based on the recorded clock signal whether the actual physiological activity or actual physiological cycle measured by the sensor unit corresponds to a desired state for providing treatment by the medical device. For doing so, the control unit may be configured to, based on the recorded clock signal, determine a parameter being indicative of the actual physiological activity or actual physiological cycle. Specifically, the parameter may be provided to quantify a characteristic of the actual physiological activity or cycle. Further, the control unit may be configured to control operation of the medical device in dependence on the parameter. Specifically, the control unit may be configured to control operation of the medical device in dependence on a comparison of the thus determined parameter with at least one reference value or at least one threshold or at least one threshold range.

More specifically, the control unit may be configured to actuate the stimulation unit if the determined parameter has reached the threshold range and to stop actuating the stimulation unit if the determined parameter lies outside the threshold range. Alternatively, the control unit may be configured to actuate the stimulation unit in dependence on a predefined actuation pattern, in particular which may not be dependent on the physiological activity or physiological cycle of the patient, if the determined parameter lies outside the threshold range and to actuate the stimulation unit in dependence on the clock signal, in particular the reference clock signal or recorded clock signal, if the determined parameter has reached the threshold range.

By such a configuration, for example, the patient may be rewarded for slow breathing by activating the non-invasive stimulation only when, for example, a breathing rate or cycle frequency of the measured and recorded breathing cycle is in the slow breathing range. For doing so, for example, the control unit may be configured to determine, as the parameter being is indicative of the characteristic of the physiological cycle, a breathing rate or mean cycle frequency based on a number of recorded preceding breathing cycles, for example based on ten preceding breathing cycles. Specifically, the control unit calculates, in particular after each passed breathing cycle, the parameter and compares it with a corresponding threshold or threshold range in order to decide whether the actual pathological cycle condition corresponds to the desired condition, i.e. the slow breathing condition.

Further, the control unit may be configured to provide information to the patient, in particular by means of the pacing unit, being indicative of a result of the comparison of the determined parameter with the predefined threshold. In this way, the medical device may provide feed-back to the patient on whether an intended reference condition is reached or not. For example, in case the medical device intends to put the patient into a slow breathing reference condition, the medical device may provide the patient with information on whether his briefing rate is too high or too low, for example by a visual and/or acoustic feedback signal.

According to one configuration, the control unit may be configured to operate the medical device, at first, in a training operating mode in which the pacing unit is actuated in dependence on the reference clock signal and the stimulation unit is controlled to stop the generation of the plurality of stimuli. Thus, in this training operating mode, the stimulation unit may be prevented from delivering stimuli to the patient.

Further, according to this configuration, the control unit may be configured to switch the operating mode of the medical device from its training operating mode into a guided operating mode when the determined parameter has reached the threshold range, wherein in the guided operating mode the control unit may be configured to actuate the pacing unit in dependence on the reference clock signal and to actuate the stimulation unit in dependence on the recorded clock signal or the reference clock signal. Further, the control unit may be configured to switch the operating mode of the medical device from its guided operating mode into its training operating mode when the determined parameter no longer reaches or lies within the threshold range, i.e. falls outside the threshold range.

Further, the control unit may be configured to switch the operating mode of the medical device from its guided operating mode into a non-guided operating mode when the medical device has been operated in its guided operating mode for a predetermined period of time, in particular during which the determined parameter continuingly reaches the threshold range, i.e. during which the determined parameter has been within the threshold range. Further, the control unit may be configured to switch the operating mode of the medical device from its non-guided operating mode into its guided operating mode, in particular for a predetermined period of time, when the determined parameter no longer reaches the threshold range.

### BRIEF DESCRIPTION OF THE DRAWINGS

The present disclosure will be more readily appreciated by reference to the following detailed description when being considered in connection with the accompanying drawings in which:

FIG. 1 is a schematic illustration of a medical device for stimulating neurons of a patient to suppress a pathologically synchronous neuronal activity;

FIG. 2 shows a diagram schematically illustrating a clock signal used in the medical device depicted in FIG. 1;

FIG. 3 schematically shows a front view of a pacing unit of the medical device depicted in FIG. 1;

FIG. 4 is a schematic illustration of a medical device according to a further embodiment;

FIG. 5 is a schematic illustration of a medical device according to a further embodiment;

FIG. 6 is a schematic illustration of a medical device according to a further embodiment;

FIG. 7 is a schematic illustration of a medical device according to a further embodiment; and

FIG. 8 schematically shows a front view of a pacing unit of the medical device depicted in FIG. 7.

### DETAILED DESCRIPTION OF PREFERRED EMBODIMENTS

In the following, the invention will be explained in more detail with reference to the accompanying Figures. In the Figures, like elements are denoted by identical reference numerals and repeated description thereof may be omitted in order to avoid redundancies.

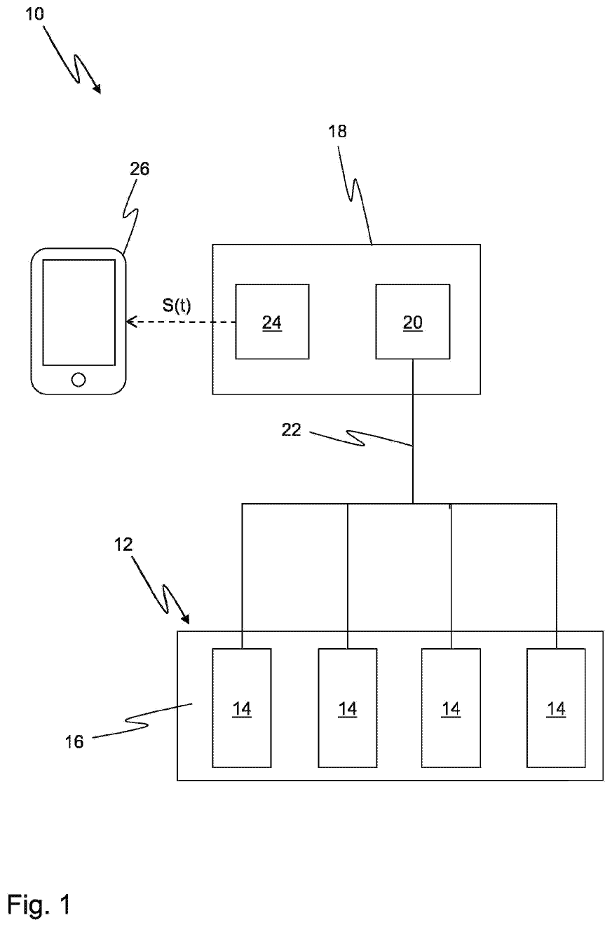

FIG. 1 schematically depicts a block diagram of a medical device 10 which is intended to be used for the therapeutic treatment of neurological or psychiatric diseases caused by a pathologically synchronous neuronal activity. Specifically, the medical device 10 is configured for stimulating neurons of a patient to suppress a pathologically synchronous activity thereof.

For acting upon the patient, the medical device 10 is equipped with a non-invasive therapeutic stimulation unit 12 which is configured to deploy a non-invasive procedure to achieve the intended therapeutic effect, i.e. the suppression of the pathologically synchronous neuronal activity of the patient's neurons. The stimulation unit 12 is configured to administer a plurality of different non-invasive stimuli to the patient's body. The plurality of stimuli is configured to suppress or to contribute to the suppression of the pathologically synchronous neuronal activity when being administered to the patient. In dependence on the application and configuration of the medical device 10, the plurality of stimuli may be configured to instantly, i.e. upon administering the first of the plurality of stimuli, suppress or contribute to the suppression of the pathologically synchronous neuronal activity. Alternatively, the plurality of stimuli may be configured to deploy the intended therapeutic effect after a part of the plurality of stimuli has been administered.

Specifically, for stimulating the neuronal population affected by the pathologically synchronous activity, the stimulation unit 12 is configured to administer the plurality of stimuli to the patient's body which, upon being sensed by respective receptors of the patient's body and guided to the patient's nerve system, may at least partially cause stimulation of the affected neuronal population. For doing so, i.e. for causing activation of the affected neuronal population, the stimulation modality, stimulation timing, stimulation characteristics, such as intensity, frequency, etc., are respectively set for each of the plurality of stimuli.

In the shown configuration, the stimulation unit 12 comprises four stimulation elements 14, but of course is not limited to this number and rather can comprise more or less than four stimulation elements 14. Each stimulation element 14 is configured to generate stimuli of varying stimulation characteristics, such as stimuli intensity and stimuli frequency. Specifically, each stimulation element 14 is constituted by an electro-mechanical actuator for converting electrical energy into a movement of a rod configured to mechanically act upon the patient's skin, thereby generating vibratory or tactile or vibrotactile stimuli. According to an alternative configuration, the stimulation elements 14 may be configured to generate stimuli of a different modality, such as acoustic stimuli, visual stimuli, electrical stimuli, electrical tongue stimuli, thermal stimuli, etc.

The stimulation elements 14 are embedded in a carrier component 16 which is configured to be detachably attached to a target area on the patient's body, in particular the patient's skin. In the shown configuration, the carrier component 16 is provided such that it can be fastened to a patient's hand. Specifically, the carrier component 16 may be provided in the form of a glove. Alternatively, the carrier component 16 may intended to be fastened to another part of the patient's body, for example, a head region or a stomach region of the patient. Accordingly, in an alternative configuration, the carrier component may be designed in the form of a headband or torso band carrying the different stimulation elements. For the sake of simplicity, the medical device 10 depicted in FIG. 1 comprises one single carrier component 16. According to a further configuration, the medical device 10 may comprise more than one carrier band 16, for example two carrier bands 16 which may be intended to be fastened to the different hands or two other parts of the patient's body.

In general, the human skin comprises mechanoreceptive afferent units capable of sensing stimuli, i.e. tactile or vibratory stimuli, which have been classified into two major categories, namely into fast adapting units (FA) and slowly adapting units (SA). The FA units respond to moving stimuli as well as the onset and removal of a step stimulus. In contrast, the SA units respond with a sustained discharge. In addition, based on the properties of their receptive fields, both categories are further classified into two different types. The fast-adapting type I (FA I) units, also referred to as RA (rapidly adapting) units, and the slow-adapting type I (SA I) units form a small, but clearly delimited receptive fields on the surface of the skin. In contrast, the receptive fields formed by the fast-adapting type II (FA II) units, also referred to as PC (Pacinian corpuscles) units, and the slow-adapting type II (SA II) are wider and have obscure borders.

Typically, the distribution and density of the different types of mechanoreceptors differs in dependence on the position on the human skin. For example, regarding the glabrous skin of the human hand, the density of FA I units is relatively high in an area of the fingertips. By contrast, the density of FA II units is relatively high in an area at the back of the fingers and the hand.

The four different types of human cutaneous mechanoreceptors respond optimally to qualitatively different stimuli. Specifically, edge stimuli and stretch stimuli are optimal for SA I and SA II mechanoreceptors, respectively. SA I units often have a rather irregular sustained discharge, whereas SA II units discharge in a regular manner, but often display spontaneous discharge in the absence of tactile stimulation. Vibratory perpendicular sinusoidal skin displacements in the range between about 30 Hz to about 60 Hz are optimal stimuli for FA I units, whereas vibratory stimuli in the range between about 100 Hz to about 300 Hz are optimal stimuli for FA II units. FA I and, especially, SA I units have a pronounced edge contour sensitivity and, hence, their response is stronger when a stimulating contactor surface which is not completely contained in the receptive field. Accordingly, to enhance the FA I responses, instead of a flat, spatially homogenous contactor surface of the stimulation element one could use a contactor surface with a spatially inhomogeneous indentation profile.

In the shown embodiment, the stimulation elements 14 are designed and configured to generate stimuli adapted to the response characteristic of FA I, FA II, SA I and/or SA II units. Each of the stimulation elements 14 is configured to generate stimuli adapted to response to at least one of the FA I, FA II, SA I and SA II units. For example, the medical device 10 may comprise stimulation elements 14 which are configured to generate stimuli which target merely one of the FA I, FA II, SA I and SA II units. In other words, these stimulation elements 14 generate stimuli which are adapted to the response characteristic of one of the FA I, FA II, SA I and SA II units. Alternatively or additionally, the medical device 10 may comprise stimulating elements 14 which are configured to generate stimuli targeting more than one of the FA I, FA II, SA I and SA II units. For example, such a stimulation element 14 may be configured to generate stimuli which are sensed by more than one of the FA I, FA II, SA I and SA II units. Alternatively or additionally, such a stimulation element 14 may be configured for being operated in different operational modes, in which different stimuli are generated which, respectively, are adapted to a response characteristic of different FA I, FA II, SA I and SA II units.

Specifically, for targeting FA I type receptors, the stimulation elements 14 may be configured to generate vibratory stimuli with a vibration frequency between 30 Hz to 60 Hz, i.e. 30 Hz, and a vibration peak-to-peak amplitude of 0.25 mm. For example, this stimulation elements 14 may be intended for being fastened to a fingertip of the patient. Further, for targeting FA II type receptors, stimulation elements 14 may be configured to generate vibratory stimuli with a vibratory frequency between 100 Hz to 300 Hz, i.e. 250 Hz, and a peak-to-peak amplitude of 2.0 mm. For example, this stimulation element 14 may be intended for being fastened to a back of a finger or hand of the patient. Further, it has been found that for sufficiently large vibration peak-to-peak amplitudes, the low-frequency vibration targeting FA I type receptors will additionally activate FA II type receptors and vice versa. Thus, by increasing the peak-to-peak amplitude, e.g. to a peak-to-peak amplitude of 3.0 mm, each of the above mentioned stimulation elements 14 may generate vibratory stimuli adapted to stimulate both FA I and FA II type receptors.

The medical device 10 further comprises a control unit 18 which is configured for selectively and intermittently actuating the stimulation unit 12, i.e. the different stimulation elements 14.

Specifically, the control unit 18 comprises an actuating unit 20 which is connected to each one of the stimulation elements 14 via connecting wires 22, through which electrical energy or control signals are selectively guided to the stimulation elements 14, respectively, so as to actuate the different stimulation elements 14 according to a desired actuation pattern.

For enabling effective treatment of the pathologically affected neuronal population, the control unit 18 is configured to control the stimulation unit 14 such that it selectively and intermittently administers the plurality of stimuli. Specifically, the different stimuli are administered to the patient's body according to an actuation and timing pattern which aims at mutually time-shifted actuations of neuronal sub-populations of varying composition, i.e. in terms of location and quantity. For doing so, an actuation sequence defined by the actuation and timing pattern is variedly adjusted by the control unit 18 during operation, for example, in view of an order of stimulus to be administered and/or a number and combination of stimuli to be simultaneously administered.

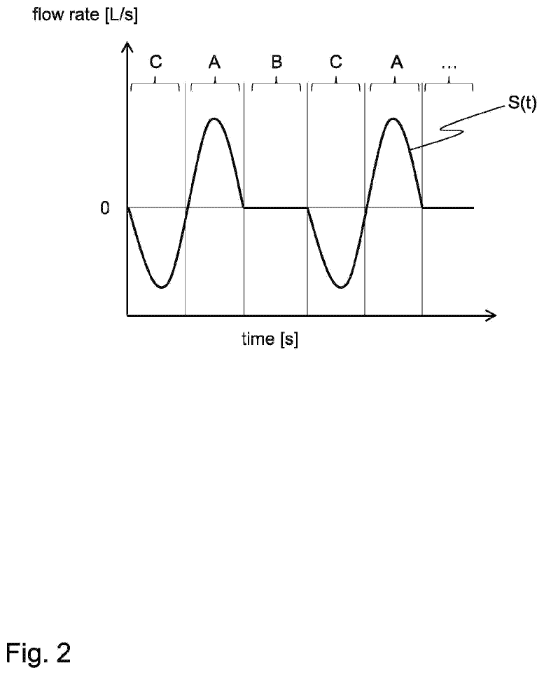

Further, the medical device 10 comprises a clock unit 24 which is configured to generate at least one periodic or cyclical clock signal which is related to a physiological activity or physiological cycle of the patient. Specifically, in the shown configuration depicted in FIG. 1, the clock signal is a reference clock signal which is denoted by reference sign “S(t)” and which relates to a reference physiological cycle in the form of a reference breathing cycle. The reference breathing cycle and thus also the clock signal S(t) comprises a repeating sequence of at least three subsequent phases A-C as depicted in FIG. 2 and as will be further described in the following. According to an alternative embodiment, the physiological cycle, in particular the breathing cycle, may comprise less or more than three subsequent phases, in particular two phases which may be constituted, for example, by an inhalation phase and an exhalation phase.

FIG. 2 depicts a diagram which exemplarily illustrates a part of the reference clock signal S(t). The abscissa of the diagram depicts the time and the ordinate of the diagram depicts an amplitude of a parameter or characteristic being indicative of a change of a patient's physiological condition according to the reference breathing cycle. As an example, in FIG. 2, a flow rate of gases being supplied to or discharged from the patient's lungs is depicted as the parameter being indicative of the reference breathing cycle. Accordingly, in FIG. 2, the reference clock signal S(t) depicts a course of the flow rate over time. It is pointed out that, instead of the flow rate, any parameter may be used which is indicative of a change of the breathing cycle, such as a parameter being indicative of a breathing-related chest wall motion.

The reference clock signal S(t) defines a repeating sequence of three subsequent phases constituted by an inhalation phase A, a breath holding phase B and an exhalation phase C. Specifically, the inhalation phase A is characterized by a positive flow rate. The exhalation phase C is characterized by a negative flow rate. The breath holding phase B is characterized by a flow rate around zero, as can be gathered from FIG. 2. Specifically, the depicted reference clock signal S(t) refers to a slow breathing cycle having a cycle frequency in the range of about 0.03 to 0.16 Hz, for example in the range of 0.07 Hz to 0.16 Hz. Within the breathing cycle, the different phases A-C may have corresponding phase lengths. Accordingly, each one of the different phases A-C of the breathing cycle may have a phase length, i.e. a duration, in the range of 2.1 s to 4.8 s. Alternatively, the exhalation phase may have a phase length that is substantially twice as long as the phase length of each one of the inhalation phase and the breath holding phase.

In the shown configuration, the reference clock signal S(t) is provided in the form of a continuous function, but may be also provided in another form. Specifically, any function or data set may qualify as the clock signal which is capable of putting a physiological activity or the respective phases of the physiological cycle, i.e. the reference breathing cycle, into relation to an associated time period.

As can be gathered from FIG. 1, the clock unit 24 is provided as an integral part of the control unit 18. Alternatively, the clock unit 24 may be provided separately from the control unit 18, i.e. in a structurally separated manner.

The control unit 18 is configured to control operation of the medical device 10 in dependence on the reference clock signal S(t) provided by the clock unit 24. Specifically, the control unit 18 is configured to control operation of a pacing unit 26 of the medical device 10 in dependence on the reference clock signal S(t). The pacing unit 26 is configured to provide guidance for a physiological activity and physiological cycle of the patient. More specifically, the pacing unit 26 is configured to instruct the patient on how to properly breathe so as to put the patient into the reference condition, i.e. into the slow deep breathing condition to which the reference breathing cycle refers. For doing so, the control unit 18 transmits the reference clock signal S(t) to the pacing unit 26 as indicated in FIG. 1 by a dashed arrow. The transmission of the reference clock signal S(t) may be performed via an electric wire or wirelessly, e.g. according to known wireless technology standards, such as Bluetooth.

Upon receiving the reference clock signal S(t), the pacing unit 26 is configured to convert or extract guiding information from the reference clock signal S(t). The intention of deriving the guiding information is to provide the reference clock signal S(t) to the patient in a form which is more accessible and easier to follow by the patient. Accordingly, the guidance for the patient provided by the pacing unit 26 is generated based on the guiding information.

In the shown configuration, the derived guiding information comprises the following mathematical function Ψ defined in the polar coordinate system as:

Ψ(t)=−½π−2πft (1)

wherein f refers to the cycle frequency of the slow breathing cycle, and t is a variable referring to time.

Further, the derived guiding information further comprises the three following polar coordinates:

φ₁=(1;−½π) (2)

φ₂=(1;− 7/6π) (3

φ₃=(1;− 11/6 π) (4

wherein φ₁ refers to a transition point between the exhalation phase C and the inhalation phase A, φ₂ refers to a transition point between the inhalation phase A and the breath holding phase B, and φ₃ refers to a transition point between the breath holding phase B and the exhalation phase C.

According to an alternative configuration or alternative operating mode of the medical device 10, the reference clock signal may be provided such that the following polar coordinates may be derived:

φ₁=(1;−½π) (5

φ₂=(1;−π) (6

φ₃=(1;− 3/2π) (7

With such a configuration, the exhalation phase C has a phase length that is substantially twice as long as the phase length of each one of the inhalation phase A and the breath holding phase B. Of course, any other suitable position or distances between the points φ₁φ₃ may be selected.

For deriving the above function and coordinates, the pacing unit 26 is configured to identify the different phases A-C of the breathing cycle in the reference clock signal S(t) and to determine their individual length. Specifically, for doing so, the pacing unit 26 may be configured to apply means of data analysis, such as filtering, in particular bandpass filtering, Hilbert transformation, transformation of the clock signal in a breathing pattern circle, statistical evaluation and/or linear or non-linear approximation, etc. Alternatively, the pacing unit 26 may be configured to compare the clock signal S(t), i.e. the course of its amplitude over time, with an upper and lower threshold. In this regard, the pacing unit 26 may associate sections of the clock signal S(t) during which the amplitude is above the upper threshold to the inhalation phase A, sections of the clock signal during which the amplitude is below the lower threshold to the exhalation phase C, and sections of the clock signal during which the amplitude is around zero or between the upper and lower threshold to the breath holding phase B.

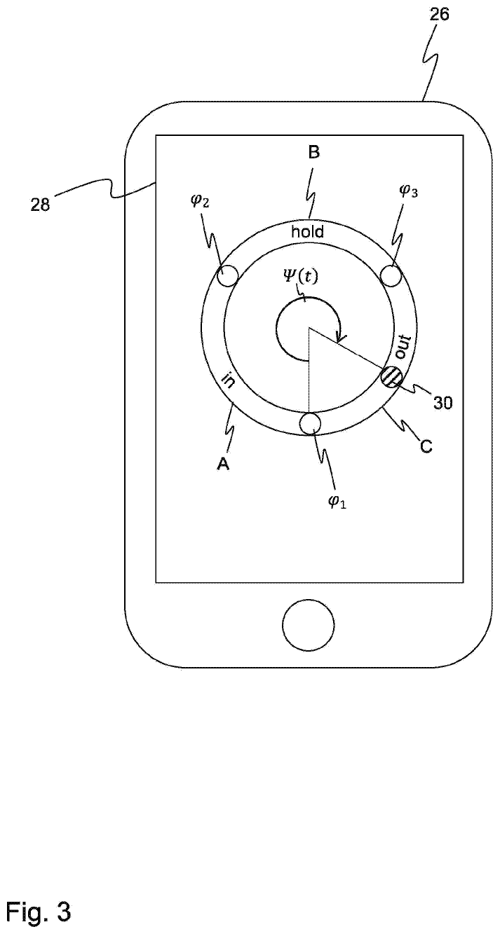

Further, the pacing unit 26 is configured to use the thus derived guiding information to instruct the patient by visualizing the guiding information as depicted in FIG. 3. Specifically, is the pacing unit 26 comprises a display unit 28 which is configured to provide non-invasive stimuli, i.e. visual stimuli, by visualizing the guiding information on a screen of the display unit 28. By doing so, the patient to be treated by the medical device 10 is informed when he or she is expected to inhale, exhale and hold his/her breath.

More specifically, the display unit 28 shows a ring through which a pacing sign 30 in the form of a ball is guided at a constant speed, i.e. at a constant angular speed of 2πf. Accordingly, the movement of the pacing ball 30 is defined by the function Ψ(t), wherein the pacing ball 30 starts at position φ₁. The ring is divided into three equally long sections, each of which is associated to one phase A-C of the breathing cycle. These sections are delimited relative to one another by small circles depicted in dotted lines which visualize the above coordinates φ₁-φ₃. Accordingly, dependent on the position of the ball 30, the patient is instructed to inhale, exhale or hold his/her breath. In this way, the medical device 10 may ensure that the patient is put into a reference state during performing the therapeutic treatment.

According to an alternative configuration, the reference clock signal S(t) may further comprise a pause phase arranged between the exhalation phase C and the inhalation phase A during which the patient is instructed to hold his/her breath. Accordingly, the pacing unit 26 may be adapted to further identify and quantify the pause phase and adapt the visualization of the derived information such that a ring is depicted which comprises four sections, each of which is associated to one phase of the breathing cycle.

As set forth above, by the pacing unit 26 according to this configuration, visual stimuli, also referred to as guidance stimuli in the present disclosure, are generated in order to provide guidance to the patient. Alternatively or additionally, for providing the guidance for the patient, at least one of acoustic stimuli, vibrotactile stimuli, vibratory stimuli, electrical stimuli, transdermal electrical stimuli transdermal magnetic stimuli, transcranial electrical stimuli and transcranial magnetic stimuli may be used.

In the shown configuration, the control unit 18, i.e. the actuating unit 20, is configured to actuate the stimulation unit 12 in dependence on a predetermined actuation pattern which is not dependent on the reference clock signal S(t).

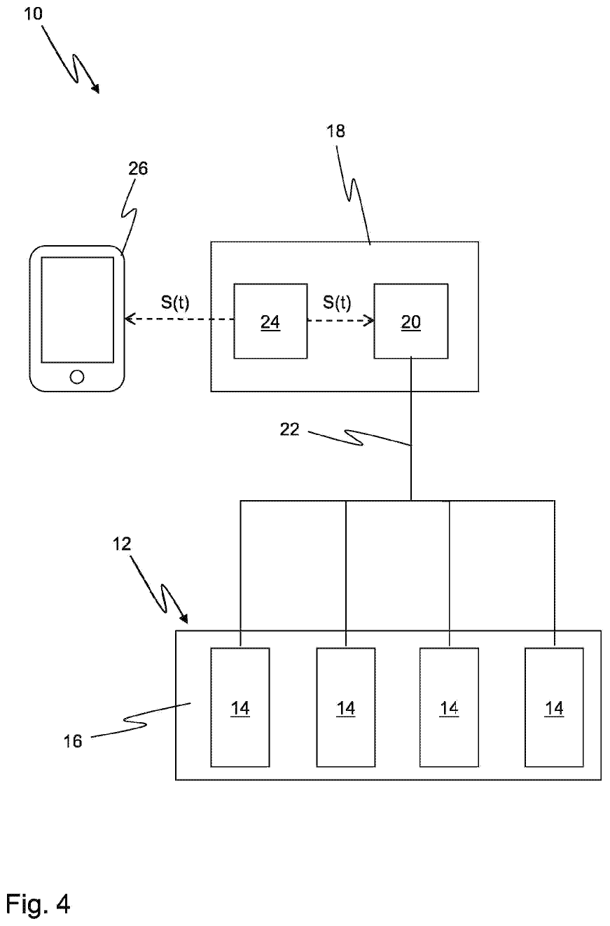

FIG. 4 shows a further embodiment of the medical device 10. Compared to the configuration depicted in FIGS. 1 to 3, the control unit 18, in particular the actuating unit 20, of the shown medical device 10 is configured to actuate the stimulation unit 12 in dependence on the reference clock signal S(t), as depicted by a further dashed arrow in FIG. 4.

More specifically, the control unit 18 is configured to actuate the stimulation unit 12 in a phase dependent manner, i.e. dependent on the different breathing phases A-C incorporated in and defined by the reference clock signal S(t). For doing so, the control unit 18, in particular the actuating unit 20, is configured to detect the different phases in the reference clock signal S(t) in a similar manner as described above in connection with the pacing unit 26. Thus, technical features which relate to the identification of phases or sections in the reference clock signal S(t) and which are described in connection with the pacing unit 26 may also be applied and relate to the control unit 18.

Based on the thus derived phases, the control unit 18, in particular the actuating unit 20, is configured to operate the stimulation unit 12 such that the stimulation unit 12 is operated in different operating modes in dependence on which phase of the breathing cycle is active and optionally in dependence on how long the phase is active. That is, if the inhalation phase A is active, the control unit 18 operates the stimulation unit 12 in a first operating mode, wherein if the breath holding phase B is active, the control unit 18 operates the stimulation unit 12 in a second operating mode, and wherein if the exhalation phase C is active, the control unit 18 operates the stimulation unit 12 in a third operating mode. At least two of the three operating modes differ from one another in at least one of a stimulation modality, a frequency of the stimuli administered, the stimulation elements which are actuated during the operating modes, a stimulation pattern, a stimulation region at the patient's body to which the stimuli are administered, an amplitude and a stimuli intensity of the stimuli administered during the operating modes.

For example, the control unit 18 may be configured to actuate the stimulation unit 12 during the inhalation phase A and the breath holding phase B and to stop the delivery of stimuli by the stimulation unit 12 during the exhalation phase C, or vice versa. Alternatively, the control unit 18 may be configured to actuate the stimulation unit 12 during the inhalation phase A and the breath holding phase B at an intensity level which is higher compared to the exhalation phase C, or vice versa.

For allowing a more fine-grained actuation of the stimulation unit 12, the control unit 18 may define actuation windows which refer to at least one operating mode, wherein the actuation windows may be scheduled and timely positioned based to the breathing cycle phases, but may not coincide with them. In other words, the actuation windows may be time-shifted relative to the breathing cycle phases and may overlap them. Further, more than one actuation window may be scheduled during a breathing cycle phase.

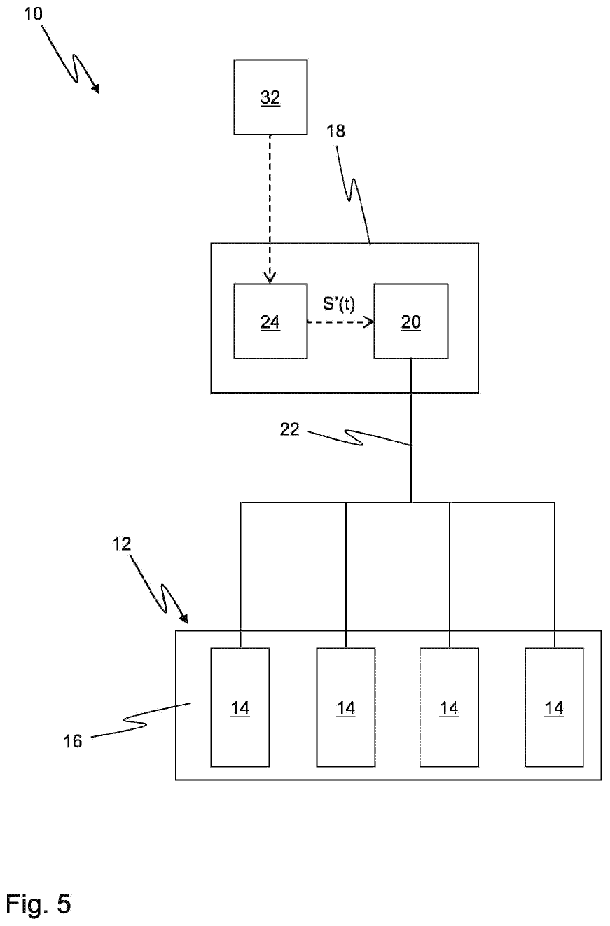

FIG. 5 shows a further embodiment of the medical device 10. Compared to the configuration depicted in FIGS. 1 to 4, the medical device 10 is not equipped with the pacing unit 26. However, the medical device 10 comprises a sensor unit 32 which is configured to monitor an actual physiological activity or an actual physiological cycle of the patient, i.e. the actual breathing cycle of the patient. Specifically, in the shown configuration, the sensor unit 32 is provided with a sensor configured to measure and record changes in the breathing cycle of the patient. For doing so, the sensor unit 32, for example, may comprise a flowmeter configured to measure and monitor a flow rate of gasses which are directed into and discharged from a patient's lungs. Such a flow meter may be comprised in a mask which is put over a patient's mouth to measure a flow rate of gasses guided into or discharge from the patient's mouth.

Further, the clock unit 24 is configured to generate a recorded clock signal which is denoted by the reference sign “S′(t)” and which is indicative of the actual physiological activity or the actual physiological cycle, i.e. the breathing cycle conducted by the patient, in dependence on monitoring data obtained by the sensor unit 32. As a general remark, it is noted that in the present disclosure reference sign “S(t)” refers to the reference clock signal and thus to the reference breathing cycle, whereas reference sign “S′(t)” refers to the recorded clock signal and thus to the actual breathing cycle performed by the patient.

In the shown configuration, the recorded clock signal S′(t) may be provided qualitatively similar to the reference clock signal S′(t) as depicted in FIG. 2.

The thus generated recorded clock signal S′(t) is then transmitted to the control unit 18, in particular the actuation unit 20, as indicated by a dashed arrow in FIG. 5. The control unit 18, in particular the actuation unit 20, is configured to actuate and operate the stimulation unit 12 in dependence of the recorded clock signal S′(t). For the actuation unit 20 it may not make a difference whether it processes the reference clock signal S(t) or the recorded clock signal S′(t). Thus, as regards the further processing of the recorded clock signal S′(t) by the actuation unit 20 and accordingly the actuation of the stimulation unit 12 based thereupon, it is referred to the above description in connection with the configuration depicted in FIG. 4.

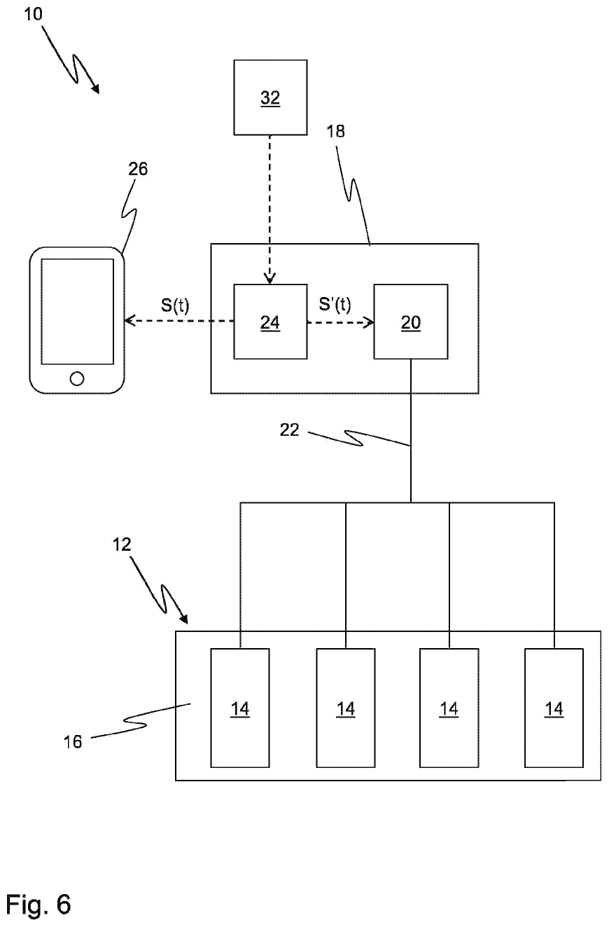

FIG. 6 shows a further embodiment of the medical device 10. Compared to the configuration depicted in FIG. 5, the medical device 10 further comprises a pacing unit 26 which is designed and configured likewise to the pacing unit 26 described in connection with the configuration depicted in FIGS. 1 to 3.

Accordingly, the clock unit 24 is configured to further generate the reference clock signal S(t) which is transferred to the pacing unit 26 and based on which the pacing unit 26 provides guidance for the patient. In one operating mode of the medical device 10, also referred to as a training mode, the control unit 18 actuates the pacing unit 26 based on the reference clock signal S(t), while the stimulation unit 12 is not active. In this mode, the patient may be trained in slow deep breathing. In a further operating mode of the medical device 10, also referred to as a stimulation mode, the control unit actuates the stimulation unit 12 based on the recorded clock signal S′(t) or the reference clock signal S(t). Additionally, the medical device 10 may be further operated in a simultaneous operating mode, in which the control unit 18 actuates both the pacing unit 26 and the stimulation unit 12. By such a configuration, the patient may be instructed to breathe according to a reference breathing cycle, while the stimulation unit 12 may be actuated in dependence on an actual physiological activity and actual physiological cycle, i.e. an actual breathing cycle of the patient.

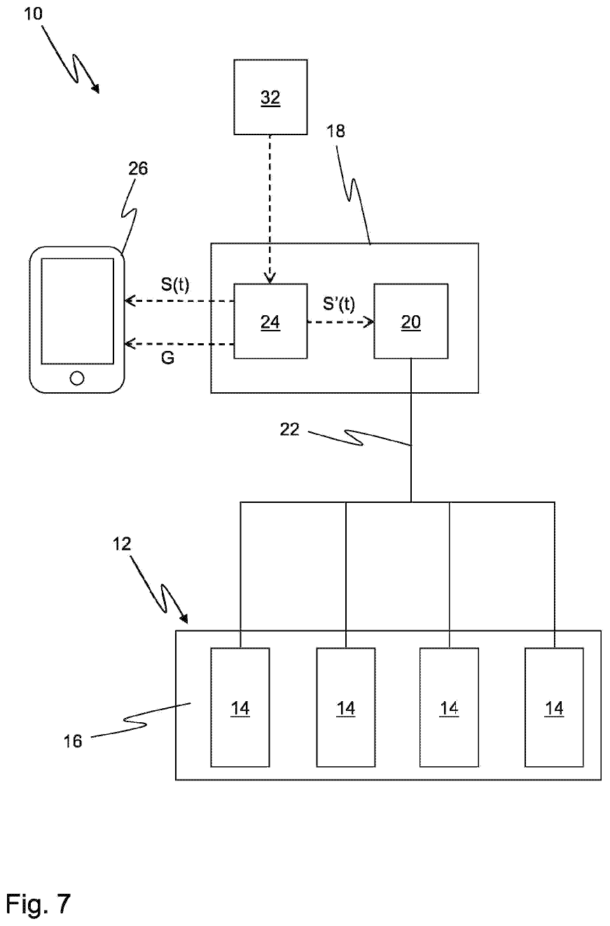

FIG. 7 shows a further embodiment of the medical device 10. Compared to the configuration depicted in FIG. 6, the control unit 18, in particular the clock unit 24, is configured to assess based on the recorded clock signal S′(t) whether the actual physiological activity or actual physiological cycle measured by the sensor unit 32 corresponds to a desired state for providing treatment by the medical device 10. For doing so, the control unit 18 is configured to instruct the patient to perform a desired breathing action and to determine a parameter G, by which an actual breathing action of the patient is compared to a desired breathing action. As such, the parameter G is indicative of a characteristic of the actual physiological activity or actual physiological cycle. For providing guidance for the patient, the control unit 18 is further configured to instruct the patient by means of the pacing unit 26 to perform slow breathing based on the reference clock signal S(t) which relates to the reference condition, i.e. the desired breathing action.

In the shown configuration, the parameter G is indicative of a goodness of paced breathing and is used to quantify whether the patient is smoothly performing slow breathing. The parameter G may be calculated after each performed breathing cycle, i.e. each time the patient has subsequently performed the breathing phases A-C. For doing so, the breathing cycle may be divided into a number of M phase bins each of which having a length or duration of:

$$Δ {T}_{M} = \frac{{T}_{C}}{M} \qquad ( 8 )$$

wherein ΔT_{M} is the duration of a phase bin; M is an integer indicating the number of bins per breathing cycle; and T_{C} is the total duration of one breathing cycle. The parameter M may be, for example, in the range of about 10 to 1000. In particular, the parameter M may be 100.

Based thereupon, the parameter G may be calculated as:

$${G}_{i} = \frac{1}{M} {\sum }_{j = 1}^{M} {h}_{correct , j} \qquad ( 9 )$$

wherein i indicates the considered breathing cycle; j indicates the j^{th} of the M different phase bins; and h_{correct,j} is a parameter indicating whether the recorded clock signal S′(t) sufficiently corresponds to the reference clock signal S(t) in the considered phase bin referred to by the control variable j. In case the recorded clock signal S′(t) sufficiently corresponds to the reference clock signal S(t) in the considered phase bin, the parameter h_{correct,j} has a value of 1, otherwise a value of 0.

Specifically, for evaluating the parameter h_{correct,j} the control unit 18 determines whether there is phase concordance between the reference clock signal S(t) and the recorded clock signal S′(t) during a considered time period, i.e. the phase bin j. That is, if the control unit 18 determines that during the phase bin j the reference clock signal S(t) and the recorded clock signal S′(t) indicate the same phase of the breathing cycle, e.g. the inhalation phase A, the control unit 18 sets h_{correct,j} to 1. However, if the control unit 18 determines that during the phase bin j the reference clock signal S(t) and the recorded clock signal S′(t) indicate different phases of the breathing cycle, e.g. the reference clock signal S(t) indicates the breath holding phase B and the recorded clock signal S′(t) indicates the inhalation phase A, the control signal sets h_{correct,j} to 0. The parameter G may therefore obtain values between 0 and 1, wherein a value of 0 indicates a phase non-concordance and a value of 1 indicates a perfect phase concordance between the reference clock signal S(t) and the recorded clock signal S′(t).

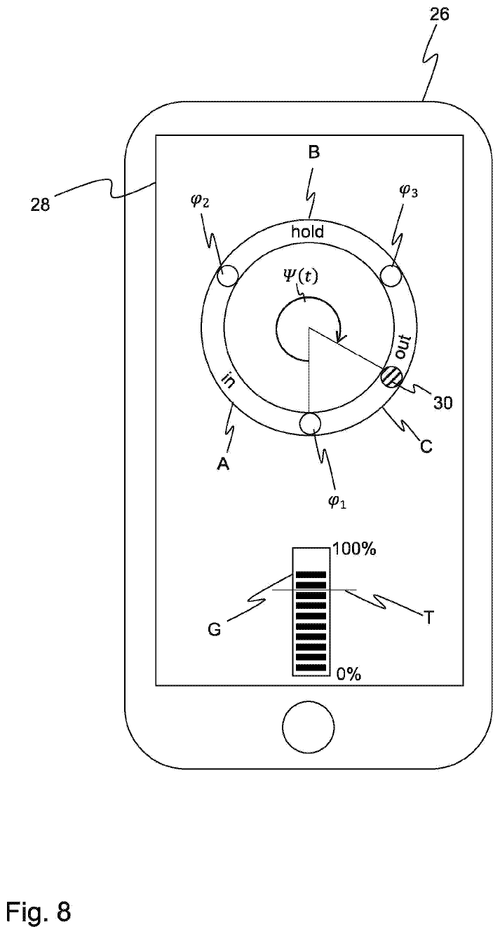

The control unit 18 is configured to transmit the determined parameter G to the pacing unit 26, as indicated by a further dashed arrow in FIG. 7. The pacing unit 26 is configured to provide information to the patient being indicative of the thus determined parameter G. Specifically, FIG. 8 depicts an example of a pacing unit 26 which is configured to provide information to the patient being indicative of the determined parameter G and a threshold T for the parameter which, as can be gathered from FIG. 8, are illustrated in the form of a bar graph. In this way, the patient gets visual feedback on the goodness of his/her paced breathing. Alternatively, the pacing unit 26 may be configured to provide an acoustic feedback to the patient.

For example, in case the determined parameter G lies outside a predetermined threshold range, the pacing unit 26 may be configured to provide an acoustic warning signal to the patient. Preferably, the predetermined threshold range extends from 0.7 to 1.0. In general, the predetermined threshold range constitutes a set of values corresponding to desired values for the parameter G and preferably lies within a range of 0.7 to 1.0. In an alternative embodiment, the predetermined threshold range may, for example, extend from 0.8 to 1.0. The volume of the acoustic warning signal may further increase as G decreases below the predetermined threshold range. Alternatively or additionally, in case the determined parameter G lies in the predetermined threshold range, the pacing unit 26 may be configured to provide an acoustic information signal, such as relaxing sounds.

Further, the control unit 18 is configured to determine a goodness of breathing criterion \tilde{G} based on the parameter G which indicates an average of the parameter G over a predetermined number K of preceding breathing cycles, e.g. of 3 or 5 or 10 or 20 breathing cycles. Specifically, the goodness of breathing criterion \tilde{G} may be calculated as:

$$\tilde{G} = \frac{1}{K} {\sum }_{k = 0}^{K} {G}_{i - k} \qquad ( 10 )$$

wherein K is an integer indicating the number of preceding breathing cycles used to calculate the criterion; and k is an integer used as a control variable.

Further, the control unit 18 is configured to control operation of the medical device 10 in dependence on a comparison of the parameter G or the criterion \tilde{G} with a reference value, i.e. a threshold value referring to a threshold range. Specifically, the reference value may refer to a value of, for example, 0.7 and may be patient-specific. As long as the determined parameter G or the criterion \tilde{G} is above the threshold value, the control unit 18 may assess that the actual physiological activity or actual physiological cycle measured by the sensor unit 32 corresponds to a desired state for providing the therapeutic treatment.

Specifically, the control unit 18 may be configured to actuate the stimulation unit 12 if the determined parameter G or the criterion G has reached the threshold range and to stop actuation of the stimulation unit 12 if the determined parameter G or the criterion \tilde{G} lies outside the threshold range, i.e. in a range below 0.7. Alternatively, the control unit 18 may be configured to actuate the stimulation unit 12 according to a predefined actuation pattern if the determined parameter G or the criterion \tilde{G} lies outside the threshold range, i.e. is below 0.7, and to actuate the stimulation unit in dependence on the reference clock signal S′(t) if the determined parameter G or the criterion \tilde{G} has reached the threshold range, i.e. is equal to or greater than 0.7.

According to a further development, the control unit 18 is configured to operate the medical device in a training operating mode in which the pacing unit 26 is actuated in dependence on the reference clock signal S(t) and the stimulation unit 12 is controlled to stop the generation of the plurality of stimuli. Further, the control unit 18 is configured to switch the operating mode of the medical device 10 from its training operating mode into a guided operating mode when the determined parameter G or the criterion \tilde{G} has reached the threshold range. In the guided operating mode, the control unit 18 is configured to actuate the pacing unit 26 in dependence on the reference clock signal S(t) and to actuate the stimulation unit 12 in dependence on the recorded clock signal S′(t).

Further, the control unit 18 is configured to switch the operating mode of the medical device 10 from its guided operating mode into its training operating mode when the determined parameter G or the criterion \tilde{G} no longer reaches the threshold range.

Further, the control unit 18 is configured to switch the operating mode of the medical device 10 from its guided operating mode into its non-guided operating mode when the medical device 10 has been operated in its guided operating mode for a predetermined period of time.

Further, the control unit 18 is configured to switch the operating mode of the medical device 10 from its non-guided operating mode into its guided operating mode when the determined parameter G or the criterion \tilde{G} no longer reaches the threshold range.

According to a further embodiment, the control unit 18 may be configured to actuate the stimulation unit 12 in dependence on a recorded clock signal S′(t) which refers to oscillations of skin microcirculation. Thus, in this configuration, the physiological cycle refers to a skin microcirculation. For doing so, the sensor unit 32 is configured to measure and record oscillations of the skin microcirculation. Specifically, the sensor unit 32 is equipped with a photoplethysmography unit, in particular an infra-red photoplethysmography unit, which is configured to be attached to the patient's skin to measure changes, i.e. oscillations, of the skin microcirculation.

Further, based on the thus obtained data by the sensor unit 32, the clock unit 24 is configured to derive a recorded clock signal S′(t) which, accordingly, refers to the oscillation of the skin microcirculation. Then, based on the thus generated recorded clock signal S′(t), the actuation unit 20 determines different phases in the signal and actuates the stimulation unit 12 in a phase-dependent manner. In this configuration, the clock unit 24 may be configured to generate a reference clock signal S(t) which refers to a reference breathing cycle. By doing so, the clock unit 24 chooses a cycle frequency of the reference breathing cycle which is different from a cycle frequency of the measured and recorded skin microcirculation. Thereafter, the control unit 24 transmits the thus generated reference clock signal S(t) to the pacing unit 26 to instruct the patient to perform breathing according to the reference breathing cycle.

In a further development, the medical device 10 depicted in FIGS. 5 and 6 may be used to empower rehabilitation training by combining the rehabilitation training with neurostimulation provided by the medical device 10. In this context it has been found that the rehabilitation training may be particularly effective if operation and actuation of the medical device 10, in particular triggering and modulation of vibrotactile stimuli generated by the stimulation elements 14, is controlled in dependence on a clock signal S′(t) related to an actual physiological activity, in particular related to phases of rhythmic or oscillatory or intermittent movements of bodily parts. To that end, the clock unit 24 may generate a movement-related clock signal S′(t), i.e. in particular related to phases of rhythmic or oscillatory or intermittent movements of bodily parts. Specifically, such a movement-related clock signal S′(t) may be an oscillatory signal which is related to an oscillatory movement of a bodily part which is subjected to rehabilitation training. For generating the movement-related clock signal S′(t), the is sensor unit 32 may be configured to measure movement of the bodily part. For example, the sensor unit 32 may comprise at least one goniometer attached to an arm of a patient in the region of the elbow and may be configured to measure angle changes of the elbow. In one configuration, the sensor unit 32 may comprise at least one twin-axis goniometer configured to measure angle changes of a bodily part in up to two movement planes. In a further development, the pacing unit 26 may be used to instruct the patient to perform the rehabilitation training and to guide the patient through a rehabilitation training.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

1. A medical device for stimulating neurons of a patient to suppress a pathologically synchronous activity of the neurons, comprising:
a non-invasive therapeutic stimulation unit configured to administer a plurality of stimuli to a patient's body which is configured to suppress the pathologically synchronous activity,
a clock unit configured to generate at least one clock signal related to a physiological activity or physiological cycle of the patient or to an activity of the patient which induces a measurable change of a physiological condition of the patient, and
a control unit configured to control operation of the medical device in dependence on the at least one clock signal.

2. The medical device according to claim 1, wherein the plurality of stimuli comprises at least one of acoustic stimuli, tactile stimuli, vibratory stimuli, vibrotactile stimuli, visual stimuli, electrical stimuli and, or thermal stimuli, and wherein the stimulation unit is configured to selectively and intermittently administer the plurality of stimuli.

3. The medical device according to claim 1, wherein the at least one clock signal comprises a repeating sequence of at least two subsequent phases.

4. The medical device according to claim 1, wherein the physiological cycle underlying the at least one clock signal is a breathing cycle which comprises at least one of an inhalation phase, a breath holding phase, an exhalation phase, or a pause phase.

5. Medical The medical device according to claim 1, wherein the clock unit is configured to generate a reference clock signal which refers to a reference condition of the patient.

6. The medical device according to claim 5, further comprising a pacing unit which is configured to provide guidance for an activity or physiological cycle of the patient, wherein the control unit is configured to operate the pacing unit in dependence on the reference clock signal.

7. The medical device according to claim 5, wherein the reference clock signal has a cycle frequency in a slow breathing range.

8. The medical device according to claim 6, wherein the pacing unit is configured to provide non-invasive guidance stimuli to the patient's body for providing guidance for an activity or physiological cycle of the patient, wherein the non-invasive guidance stimuli comprises at least one of visual stimuli, acoustic stimuli, tactile stimuli, vibratory stimuli, vibrotactile stimuli, electrical stimuli, transdermal electrical stimuli transdermal magnetic stimuli, transcranial electrical stimuli, transcranial magnetic stimuli, or electrical tongue stimuli, and wherein the non-invasive guidance stimuli are configured to guide or instruct the patient to perform an activity.

9. The medical device according to claim 6, wherein the control unit is configured to actuate the stimulation unit in dependence on a predetermined actuation pattern.

10. The medical device according to claim 1, further comprising a sensor unit configured to monitor an actual activity or an actual physiological cycle of the patient, wherein the clock unit is configured to generate a recorded clock signal being indicative of the actual activity or the actual physiological cycle in dependence on monitoring data obtained by the sensor unit.

11. The medical device according to claim 1, wherein the control unit is configured to actuate the stimulation unit in dependence on the at least one clock signal.

12. The medical device according to claim 11, wherein the control unit is configured to operate the stimulation unit in a first operating mode during a first phase of the at least one clock signal and in a second operating mode which differs from the first operating mode during a second phase of the at least one clock signal, and wherein the first operating mode and the second operating mode of the stimulation unit differ in at least one of a stimulation modality, a frequency of the stimuli to be administered, stimulation elements to be actuated, stimulation amplitude, a stimulation pattern, a stimulation region at the patient's body to which the stimuli are to be administered, or stimuli intensity.

13. The medical device according to claim 10, wherein the control unit is configured to, based on the recorded clock signal, determine a parameter quantifying a characteristic of the actual activity or cycle, and wherein the control unit is configured to control operation of the medical device in dependence on a comparison of the parameter with a threshold or a threshold range.

14. The medical device according to claim 13, wherein the control unit is configured to actuate the stimulation unit if the parameter has reached the threshold range and to stop actuation of the stimulation unit if the determined parameter lies outside the threshold range.

15. The medical device according to claim 13, wherein the control unit is configured to provide information to the patient being indicative of a result of the comparison of the parameter with the threshold.

16. Medical The medical device according to claim 13, further comprising a pacing unit which is configured to provide guidance for an activity or physiological cycle of the patient, wherein the clock unit is configured to generate a reference clock signal which refers to a reference condition of the patient, wherein the control unit is configured to operate the medical device in a training operating mode in which the pacing unit is actuated in dependence on the reference clock signal and the stimulation unit is controlled to stop a generation of the plurality of stimuli, wherein the control unit is configured to:
switch an operating mode of the medical device from a training operating mode into a guided operating mode when the parameter has reached the threshold range, wherein in the guided operating mode the control unit is configured to actuate the pacing unit in dependence on the reference clock signal and to actuate the stimulation unit in dependence on the recorded clock signal;
switch the operating mode of the medical device from the guided operating mode into the training operating mode when the parameter no longer reaches the threshold range;
switch the operating mode of the medical device from the guided operating mode into a non-guided operating mode when the medical device has been operated in the guided operating mode for a predetermined period of time; and
switch the operating mode of the medical device from the non-guided operating mode into the guided operating mode when the determined parameter no longer reaches the threshold range.

17. The medical device according to claim 5, wherein, the reference condition of the patient is a reference activity or a reference physiological cycle of the patient.

18. The medical device according to claim 7, where the slow breathing range is in a range of 0.07 Hz to 0.16 Hz.

19. The medical device according to claim 9, wherein the predetermined actuation pattern is not dependent on the reference clock signal.

20. The medical device according to claim 11, wherein the clock unit is configured to generate a reference clock signal which refers to a reference condition of the patient, wherein the clock unit is configured to generate a recorded clock signal being indicative of an actual activity or an actual physiological cycle of the patient, and wherein the control unit is configured to actuate the stimulation unit in dependence on the reference clock signal or the recorded clock signal.

21. The medical device according to claim 13, wherein the control unit is configured to actuate the stimulation unit according to a predefined actuation pattern if the parameter lies outside the threshold range and to actuate the stimulation unit in dependence on the recorded clock signal if the parameter has reached the threshold range.
